(19) United States
(12) Patent Application Publication
(10) Pub. No.: US 2010/0099894 A1
(43) Pub. Date: Apr. 22, 2010

(54) Method for the Synthesis of Cyclic Acetals by the Reactive Extraction of a Polyol in a Concentrated Solution

(76) Inventors: Jean-Luc Dubois, Millery (FR); Sara Iborra Chornet, Valencia (ES); Alexandra I. Lucienne Velty, Valencia (ES); Avelino Corma Canos, Valencia (ES)

Correspondence Address: K&L Gates LLP, 1900 MAIN STREET, SUITE 600, IRVINE, CA, 92614-7319, US

(21) Appl. No.: 12/445,030

(22) PCT Filed: Oct. 8, 2007
(86) PCT No.: PCT/FR2007/052091

§ 371 Date: Nov. 9, 2009

(30) Foreign Application Priority Data
Oct. 9, 2006 (FR) 0654153

(51) Int. Cl. C07D 319/06; C07D 317/10

(52) U.S. Cl. 549/372; 549/449

(57) ABSTRACT

A method for the synthesis of cyclic acetals comprises reacting at least one carbonyl-function compound selected from aldehydes, ketones, and/or linear acetals, on a polyol in a concentrated aqueous solution exceeding 20 wt % in a reactor containing an acidic catalyst. The carbonyl-function compound is selected so that the cyclic acetal obtained has a water solubility lower than 20000 mg/kg. During the catalytic reaction for the cyclic acetal synthesis, at least one portion of the organic phase containing the cyclic acetal is separated. The acidic catalysis is either homogeneous when using a water-soluble strong acid, or heterogeneous when using a solid acid such as a resin, a zeolite, or any appropriately acidified solid. The extractive reaction method can be used for obtaining high conversions and selectivity.

### BACKGROUND

Synthesis of acetals is described at length in “Methods for the Preparation of Acetals from Alcohols or Oxiranes and Carbonyl Compounds” by F. A. J. Meskens at pages 501 to 522 of the “Synthesis” Review July 1981 (Georg Thieme Verlag-Stuttgart-New York).

Cyclic acetals produced following the method of the present disclosure are formed by the simultaneous reaction of the aldehyde and/or ketone on two hydroxyl functions of the polyol. The proximity of the two hydroxyl functions entails the formation of a cycle comprising generally, although not exclusively, 5 to 6 atoms of which 2 are oxygen. This reaction is a balanced and reversible reaction and is accompanied by water formation.

Some cyclic acetals of glycerol can be synthetised following several methods. Thus, the acetal of glycerol and of acetone (RN 100-79-8) is also known under the name of Solketal. It is a solvent with a boiling point of 188-189° C. (and of 82° C. at 10 mmHg), a melting point of −26.4° C. and a flash point of 80° C., and it is miscible in water.

The synthesis of this glycerol acetal following a technique of reactive separation has been described elsewhere. A technology of catalytic distillation has been used by Kvaerner Process Technology Ltd, J. S. Clarkson et al. Organic Process Research & Development, 2001, 5, 630-635.

Glycerol formal is another glycerol acetal that is commercially available from the company Lambiotte. It exists in the form of 2 isomers, dioxane (6-atom cycle) and dioxolane (5-atom cycle).

Patent FR 2869232 is related to pharmaceutical or cosmetic excipients based on cyclic acetals including glycerol acetals. Thus, it provides exemplary methods for synthesising acetals obtained from propanaldehyde, butyraldehyde, and pentanal. This patent advocates the use of light aldehydes in order to maintain the produced aldehyde and acetal in the aqueous phase.

U.S. Pat. No. 5,917,059 describes a method for synthesising light glycerol acetals, through the continuous distillation of an excess of aldehyde or ketone carrying the water produced by the reaction and by the continuous contribution of the aldehyde or ketone containing less than 1% water.

The works of R. R. Tink and A. C. Neish describe a reactive extraction of glycerol in the form of glycerol acetal (Can. J. Technol. 29 (1951) 243-249; ibid 29 (1951) 250-260; ibid 29 (1951) 269-275). Therein, several aldehydes or ketones are tested for extracting glycerol from diluted aqueous solutions, and concluded that butyraldehyde was the one that allowed the strongest acetal concentration in the organic aldehyde or ketone phase. However, butyraldehyde, just like its glycerol acetal, is also soluble in water, which for the efficiency of the method requires separating the acetal from the aqueous solution.

Patent application JP 10-195067 describes a method for synthesising glycerol acetals. According to this patent application, an aqueous solution of glycerol containing 30% to 80% weight in glycerol, or of hydrated glycerol containing 20% to 1% weight in water (in other words 80% to 99% of weight in glycerol) is put into contact with an aldehyde or ketone in the presence of an acidic catalyst, in a solvent with a boiling point of generally less than 150° C. and having a boiling point lower than that of the aldehyde or ketone, and whose role is to eliminate the water present in the environment, coming either from the initial solution or from the reaction, through azeotropic distillation.

The synthesis of acetals being reversible, to obtain high yields, the balance needs to move in favour of the formation of synthesised products. Several methods illustrated by the references cited above, can be used to alter this balance. One can mention: 1) use of an excess of reagent with the inconvenience of having to separate it upon conclusion of the reaction and leading to a weak conversion of this reagent, 2) use of a solvent that can carry the water that is formed with it presenting the disadvantage of increasing the cost of raw materials and requiring an additional stage of separation, or 3) use of reactive or catalytic separation such as distillation, but that cannot be applied to all reactions due to the presence of azeotropes. This last type of procedure, more recent than the previous ones, is the one used by the company Lambiotte in order to produce Methylal which is described in Swiss patent CH 688041.

### SUMMARY

The present description provides methods for the continuous industrial manufacture of cyclic acetals by extraction of polyols, and in particular, of glycerol contained in aqueous solutions that does not have the disadvantages of the procedures mentioned above.

The present disclosure generally relates to methods for the synthesis of cyclic acetals by the reaction of at least one carbonyl-function compound selected from aldehydes, ketones and/or linear acetals on a polyol in a concentrated aqueous solution, characterised in that it is carried out in a reactor containing an acidic catalyst and in that the carbonyl-function compound is selected so that the cyclic acetal obtained has a water solubility of lower than 20,000 mg/kg at room temperature, and that simultaneously with the catalytic reaction for the synthesis of the cyclic acetal, at least one portion of the organic phase containing the cyclic acetal is separated by extraction from the continuous aqueous phase in the reactor.

The present disclosure further generally relates to methods for the synthesis of cyclic acetals based on concentrated solutions of polyols. In other words, solutions containing initially at least 20% by weight of the polyol in water, and alternatively more than 40% by weight. The high content of polyols which constitutes an excess of polyol reagent will allow the total conversion of the other reagent, the carbonyl-function compound and in particular the aldehyde and/or the ketone in the reactor.

### BRIEF DESCRIPTION OF THE DRAWINGS

FIG. 1 graphically represents the relative concentration of glycerol in the aqueous phase over time.

### DETAILED DESCRIPTION OF EXAMPLE EMBODIMENTS

The present description generally relates to methods for synthesizing cyclic acetals by reactive extraction of polyols contained in aqueous phase by reacting aldehydes and/or ketones on the polyols leading to the formation of cyclic acetals of the polyols which are insoluble in the aqueous phase.

By way of example, the polyols that can be used in the methods of the present disclosure include: glycerol, ethylene glycol, propane diole, and/or propane diole, and butanediol.

Further by way of example, aldehydes that can be used in the methods of the described herein include but are not limited to: butyraldehyde, n-heptanaldehyde, 2-ethylhexanaldehyde, valeraldehyde, glyoxal, glutaraldehyde, furfuraldehyde, isovaleraldehyde, acroleine, crotonaldehyde, decanaldehyde, 2-ethylbutaraldehyde, hexanaldehyde, isobutyraldehyde, isodecanaldehyde, laurinaldehyde, 2-methylbutyraldehyde, nonanaldehyde, octanaldehyde, pivalaldehyde, tolualdehyde, benzaldehyde, tridecanaldehyde, and undecanaldehyde.

Still further by way of example, ketones that can be used in the methods of the present description include in particular: acetone, methylethyl ketone, diethyl ketone, methyl isopropyl ketone, methylisobutyl ketone, diisobutyl ketone, diisopropyl ketone, mesityl oxide, butanedione, cyclohexanone.

In order to obtain cyclic acetals with water solubility of lower than 20000 mg/kg at room temperature, it is preferable to choose as reagents carbonyl-functionalized compounds with their own solubility in aqueous solution of lower than 20000 mg/kg and alternatively lower than 10,000 mg/kg at room temperature.

Data regarding the solubility in water of chemical compounds is provided in particular in: Yaw's Handbook of Thermodynamic and Physical Properties of Chemical Compounds, 2003, Knovel.

Selected aldehydes or ketones are generally chosen from among aldehydes or ketones comprising 4 to 12 carbon atoms, and alternatively 5 to 9. Among aldehydes or ketones particularly suited to the method of the present disclosure, aldehydes may include benzaldehyde (6570 mg/kg), heptanals and in particular n-heptanal (1516 mg/kg), hexanal (5644 mg/kg), pentanal (11700 mg/kg), n-octanal (370 mg/kg); and ketones may include acetophenone (6842 mg/kg), benzophenone (136.7 mg/kg), diisobutyl ketone (2640 mg/kg) and diisopropyl ketone (5700 mg/kg).

According to the present disclosure, in one exemplary embodiment heptanaldehyde (n-heptanal) is used because it can be obtained from biomass. Heptanaldehyde is obtained, for example, from the thermal cracking of the methyl ester of castor oil.

When the carbonyl-function compound is a linear acetal, it is possible to choose, for example, acetals from among light alcohols and heavy aldehydes in order to produce acetals with poor solubility in water. Light alcohols can be selected from methanol, ethanol, propanol, and the heavy aldehydes can be the same as those mentioned above. Further, for example, the linear acetals may be selected from malonaldehyde bis (diethylacetal) (CAS RN 122-31-6), 1.1. diethoxycyclohexane (CAS RN 183-97-1), phenylacetaldehyde dimethylacetal (CAS RN 101-48-4), benzaldehyde dimethylacetal (CAS RN 1125-88-8), 1.1 dimethoxyheptanalacetal (CAS RN 10032-55-0), and 1.1 dimethylhexanalacetal (CAS RN 1599-47-9).

The catalyst of the reaction is an acidic catalyst in either solid form, as a heterogeneous catalyst, or in liquid form, as a homogeneous catalyst. Alternatively, the reactor is an agitated reactor in the case of a homogeneous catalyst.

Liquid catalysts may be selected from among those that catalyse the reaction between an alcohol and an aldehyde or a ketone, and include but are not limited to: hydrochloric acid, nitric acid, sulphuric acid, methane sulphonic acid, paratoluenesulphonic acid, triflic acid, and oxalic acid. In one example embodiment, acids soluble in the aqueous phase will be selected.

Acid solids that can be used as catalysts include but are not limited to: acid-ion exchanging resins, acid resins, natural or synthetic zeolites such as mordenite, Y zeolite, ZSM5, H-Beta, Montmorillonite, or silica-aluminas, NAFION®, and NAFION® composites or finally supported heteropolyacids, the chlorides of lanthanide, iron, zinc or titanium or the catalysers appearing in the list provided in the article by F. A. J. Meskens cited above. ZSM5 is an aluminosilicate zeolite mineral belonging to the pentasil family of zeolites. ZSM5's chemical formula is NaₙAlₙSi₉₆₋ₙO₁₉₂.16H₂O (0<n<27).

A further option is where the acid catalyst consists of an acid phase with a Hammett H₀ acidity lower than +2 and alternatively lower than 0.

Hammett acidities of acid solids are set out in the table below:

| | H₀ values of different acids |
|---|---|
| SiO₂/MgO | −1 to −3 |
| H₃PO₄/SiO₂ | −3 to −6 |
| Sulphonated resins | 0 to −4 |
| AMBERLYST ® 15 | −2 |
| Beta zeolite | −7 to −10 |
| Montmorillonite | −3 to −5 |
| NAFLON ® | −10 to −14 |
| Zeolites | −7 to −9 |

The Hammett acidity of a solid is defined in the article by K. Tanabe et al in “Studies in Surface Science and Catalysis”, Vol 51, 1989, chapters 1 and 2. Hammett acidity is determined by amino titration with the help of indicators or by absorption of an alkali in a gas phase. Hammett acidity is one of several scales of the acidity of solids. The literature provides correlations between the various acidity scales.

Acid solids that may be suitable according to the present methods are natural or synthetic chalky materials or acid zeolites; mineral supports, such as oxides, coated in inorganic acids, mono, di, tri or polyacids; oxides or mixed oxides or again heteropolyacids.

The catalyst can consist of an acid phase chosen from among acid resins such as AMBERLYST® (in particular AMBERLYST® 15 and 36), zeolites, NAFLON® composites (based on the sulphonic acid of fluorinated polymers), chlorinated aluminas, phosphotungstic and/or silicotungstic acids and acid salts, and different solids of the metallic oxides type such as tantalum oxide Ta₂O₅, niobium oxide Nb₂O₅, aluminium Al₂O₃, titanium oxide TiO₂, zirconium ZrO₂, tin oxide SnO₂, silica SiO₂ or silico-aluminate SiO₂—Al₂O₃, impregnated in acid functions such as borate, BO₃, sulphate SO₄, tungstate WO₃, phosphate PO₄, silicate SiO₂, or molybdate MoO₃.

In the methods of the present disclosure, the cyclic acetals may be obtained for example according to the following reaction mechanisms:

1)

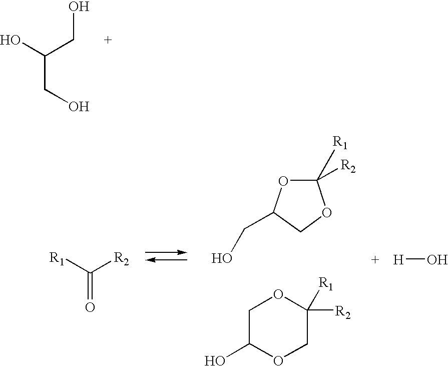

R₁ and R₂, identical or different, are either hydrogen, or a linear or branched alkyl radical, saturated or unsaturated, comprising if necessary a second function of a cetonic or ether type, or a cyclic or aromatic radical, containing 1 to 22 carbon atoms.

2)

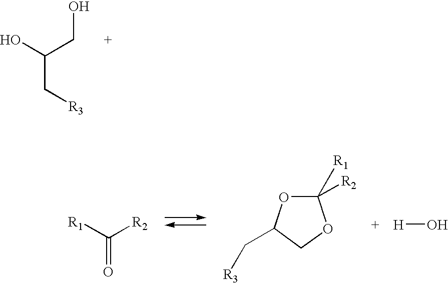

R₃ is either hydrogen, or a linear or branched alkyl radical, saturated or unsaturated, cyclic or aromatic, containing 1 to 22 carbon atoms, R₁ and R₂ corresponding to the definition above.

3)

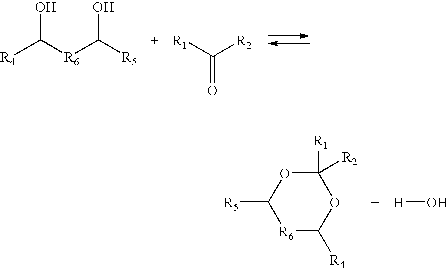

wherein R₄ and R₅ represent either hydrogen, or linear or branched alkyl radicals, saturated or unsaturated, or cyclic or aromatic radicals, containing 1 to 22 carbon atoms. R₆ is a linear or branched alkyl grouping, saturated or unsaturated, cyclic or aromatic, having 0 to 22 carbon atoms, R₁ and R₂ responding to the definition above.

In one example embodiment, when R₁ and/or R₂ are alkyl or alkenyl radicals, the total number of carbon atoms in R₁+R₂ is equal to or more than 4 and alternatively more than 6.

In another example embodiment, when R₁ and/or R₂ comprise a second carbonyl-type function (aldehyde or ketone) bi and/or tri cyclic acetals are obtained.

### 4) Through Implementation of Transacetalisation

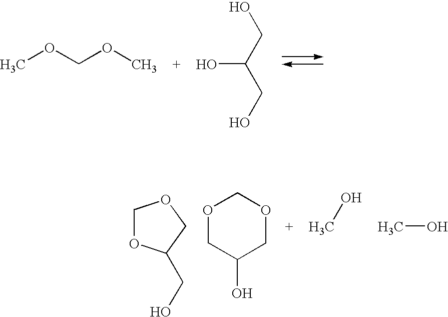

or more generally

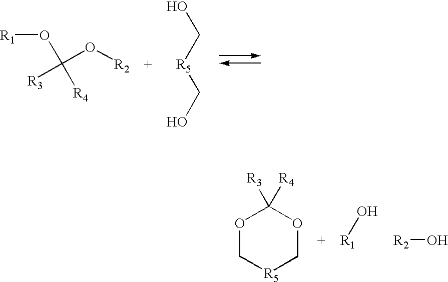

wherein R₁, R₂, R₃ and R₄ are linear or branched alkyl groupings, saturated or unsaturated, cyclic or aromatic, having 1 to 22 carbon atoms, and R₅ is either CH₂, CHOH, or a linear or branched alkylene grouping, saturated or unsaturated, cyclic or aromatic, having 0 to 22 carbon atoms.

The reactions described herein can be carried out at a temperature close to room temperature, which will generally be between 5 and 200° C. and at a pressure generally between 100 kPa and 8000 kPa. The reaction environment may need to be strongly agitated in order to favour the contact between the organic fraction containing the aldehyde/ketone insoluble in water and the aqueous fraction containing the glycerol.

In an alternative embodiment of the present methods, the synthesis of cyclic acetals by extraction of the polyol in solution is carried out in several consecutive reaction stages (extractive in respect of the polyol). In the first stage, part of the polyol of the concentrated solution is extracted with the help of a weak concentration of aldehyde/ketone. Following transfer to the next stage, the polyol solution is thus more diluted and the polyol is made to react with a higher concentration of aldehyde/ketone, this operation being repeated as many times as necessary with a progressive increase in the relative concentration of aldehyde/ketone.

The duration of the reaction in each extraction stage will depend on the kinetics of the reaction and consequently on the concentration in each reagent. Similarly, the temperature and pressure can be adjusted independently in each reaction stage. A higher temperature makes it possible to accelerate the reactions in particular, but also shifts the balances. This is why it is preferable to use higher concentrations in aldehyde/ketone in the successive stages.

According to an alternative mode of the present disclosure, the first reaction stage works at a higher temperature, and/or with a shorter reaction time than the subsequent stages.

A reaction stage generally encompasses a zone of mixing the reagents, a zone of reaction, and a zone of separation of the aqueous phase from the organic phase. In certain reactor configurations one or more of the zones can be confused.

The methods of the present disclosure are particularly suited to the treatment of aqueous solutions containing impurities in the solution. For example, the glycerol quality known as Raw Glycerine contains glycerol in aqueous solution, but also sodium or potassium salts, in the form of sodium or potassium chloride, or even sodium or potassium sulphate, salts that originate from the transesterification catalyst of vegetable oils or animal fats having allowed for example the formation of biodiesel. If it is associated to a biodiesel production unit, the method of the present disclosure can allow direct treatment of the Raw Glycerine at glycerol concentrations of more than 20% weight. By then, using the homogenous catalyst with methane sulphonic acid, paratoluenesulphonic acid, or alternatively sulphuric or hydrochloric acid, the aqueous effluents of the reaction can be returned to the biodiesel unit's stage of effluent neutralisation or transesterification.

After leaving the reactor, the products of the reaction are mixed. The cyclic acetal is then separated from the organic phase through any separation technique that an expert in the field is familiar with. For its part, the aqueous phase can be easily recycled.

The method of reactive separation helps to resolve several difficulties associated to the prior art. In particular, it allows having a continuous method, whereas often the synthesis of acetals is carried out following discontinuous methods. Another advantage of the method consists of the mixture of reagents not being originally necessarily free of water, to the extent that it is separated continuously in the course of the process. In discontinuous reactors, wherein water is a product of the reaction, the presence of water shifts the balance towards the formation of reagents leading to decreased yields. In order to avoid this inconvenience, one is obliged to use anhydrous reagents.

In the methods of the present disclosure, it is possible to use a mixture of reagents containing 0 to 80% weight in water, alternatively 0 to 60% in water and further alternatively 1 to 50%.

Cyclic acetals can undergo subsequent transformations according to the final purpose for their use. For example, cyclic acetals may be transformed through transacetalisation into another cyclic acetal adapted to the application. Cyclic acetals can be subjected to hydrolysis if the product sought after is a pure glycerol, it being possible to reuse the resulting aldehyde or ketone in the process.

The cyclic acetals of glycerol have many applications, thus one could mention the preparation of cross-linkers, solvents, synthesis intermediaries, and fuel additives and can be found in numerous fields such as pharmacy and cosmetic products, bioregulators in agro-chemistry, biodegradable polymers (in particular in the formulation of chewing gum), and the preparation of glycerides.

In cyclic acetal synthesis reactions, the by-products of the reaction can be ethers obtained through dehydration of the alcohol (or polyol), or polyacetals obtained through the consecutive reaction of the aldehyde/ketone on the acetal. The formation of ethers generally occurs when increasing the reaction temperature with a view to shifting the balances. In the reactive separation according to the present disclosure, the shifting of balances is provided by eliminating of one of the products of the reaction and not by increasing the temperature, thus reducing the appearance of said by-products.

The methods of the present disclosure thus make it possible to obtain not only high conversions, but also high selectivities.

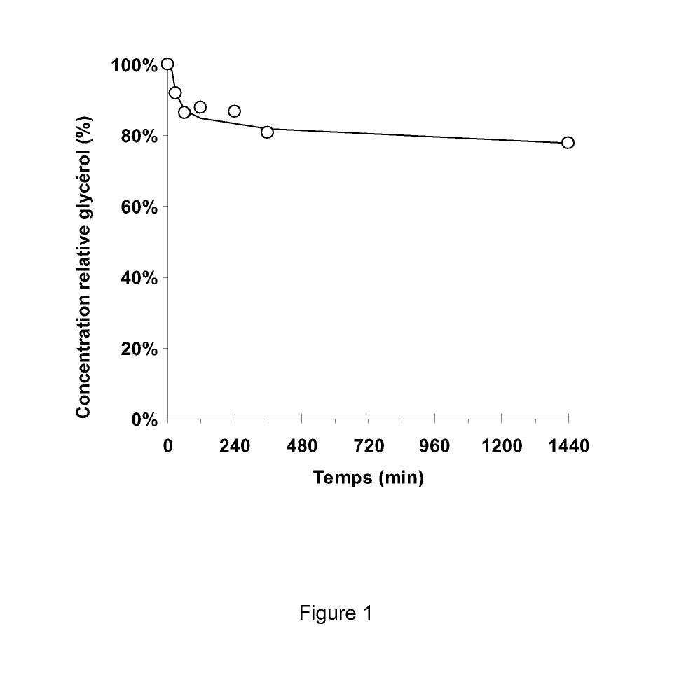

The methods of the present disclosure are illustrated by the following examples provided by way of illustration but not limitation, in addition to FIG. 1 showing the relative concentration of glycerol in the aqueous phase over time.

### Examples

### Example 1

This example illustrates the reactive extraction of glycerol through acetalisation with heptanaldehyde, using hydrochloric acid as catalyst, and with a heptanaldehyde/glycerol molar ratio of 1.

An aqueous solution of glycerol (at 60% weight of glycerol, 40% weight of water) containing 10 mmoles of glycerol (in other words 0.920 g) is mixed with 10 mmoles of heptanaldehyde (in other words 1.14 g) in order to have a heptanal/glycerol ratio of 1. A hydrochloric acid solution at 35% is added to the aqueous solution of glycerol+heptanaldehyde, in a proportion of 15% in relation to the glycerol. The mixture is then heated to 40° C. for 5 hours under agitation. Next the two phases are separated by decantation, and the organic phase is washed with water, until obtaining a neutral pH. The solution obtained is then dried under anhydrous MgSO₄, and then concentrated by evaporation in vacuum. The obtained product has been analysed by RMN and mass spectrometry and quantified by chromatographic analysis. The yield in acetal of the obtained glycerol is of 43% weight in relation to the heptanaldehyde.

### Example 2

The cyclic acetal of glycerol and heptanaldehyde is obtained as in the preceding example, but using a heptanaldehyde/glycerol molar ratio of 0.6 with a mass of glycerol of 1.47 g (16 mmoles) instead of 0.920 g. In this case, the yield of the synthesis is 73% weight in relation to the heptanaldehyde.

### Example 3

The cyclic acetal of glycerol and heptanaldehyde is obtained as in example 1, but using a heptanaldehyde/glycerol molar ratio of 0.33, and with a mass of glycerol of 2.76 g (30 mmoles) instead of 0.920 g. In this case, the yield of the synthesis is 79% weight.

These 3 examples illustrate the formation of the cyclic acetal with excellent yields and show that it is preferable to have weak aldehyde and/or ketone/glycerol ratios and therefore to carry out the extraction in several successive stages.

### Example 4

Reactive extraction of glycerol through acetalisation with heptanaldehyde, using an AMBERLYST® 36 resin as the heterogeneous catalyst, and with a heptanaldehyde/glycerol molar ratio of 0.33.

An aqueous solution of glycerol at 60% weight in glycerol, 40% weight in water containing 30 mmoles of glycerol (or 2.76 g) is mixed with 10 mmoles of heptanaldehyde (or 1.14 g) in order to have a heptanal/glycerol molar ratio of 0.33. An AMBERLYST® 36 resin is added to the aqueous solution of glycerol+heptanaldehyde, in a proportion of 15% in relation to the glycerol. The mixture is then heated to 40° C. for 5 hours under agitation.

The catalyst is then filtered. Next the two liquid phases are separated through decantation. The aqueous phase is extracted several times using dichloromethane. The dichloromethane solution is then added to the organic phase, which is then concentrated by evaporation in vacuum. The product obtained has been analysed by RMN and mass spectrometry and quantified by chromatographic analysis. The obtained yield in glycerol acetal is 80% weight in relation to the heptanaldehyde.

### Example 5

Example 5 is carried out as Example 4, but using a more diluted aqueous solution of glycerol (40% weight in glycerol instead of 60% weight). The yield in acetal is then 71% in weight in relation to the heptanaldehyde.

These two examples 4 and 5, show that it is preferable to use concentrated solutions rather than diluted solutions.

### Example 6

Reactive extraction of glycerol by acetalisation with heptanaldehyde, using as a heterogeneous catalyst a Beta Zeolite with a Si/Al atomic ratio of 13, and with a heptanaldehyde/glycerol molar ratio of 0.33.

An aqueous solution of glycerol at 60% weight in glycerol and 40% weight in water containing 30 mmoles of glycerol (or 2.76 g) is mixed with 10 mmoles of heptanaldehyde (or 1.14 g) in order to achieve a heptanal/glycerol molar ratio of 0.33. A Beta Zeolite with a Si/Al atomic ratio of 13 is added to the aqueous solution of glycerol+heptanaldehyde, in a proportion of 20% in relation to the glycerol. The mixture is then heated to 40° C. for 5 hours under agitation.

The catalyst is then filtered. Next, the two liquid phases are separated by decantation, and the aqueous phase is extracted several times using dichloromethane. The solution of dichloromethane is then added to the organic phase, which is then concentrated by evaporation in vacuum. The product obtained has been analysed by RMN and mass spectrometry, and quantified through chromatographic analysis. The obtained yield in glycerol acetal is 42% weight in relation to the heptanaldehyde.

This example illustrates the impact of the choice of catalyst on the yield of the synthesis.

### Example 7

Study of the composition of the organic phase and the aqueous phase in the course of the acetalisation reaction between the glycerol and the heptanaldehyde.

To 5 g of heptanaldehyde 3 equivalents of glycerol are added (11.92 g) in an aqueous solution at 60% weight, in other words 40% weight in water (7.85 g). An aqueous solution of hydrochloric acid at 35% is then added to the mixture (0.75 g), 4% in weight in relation to the glycerol. The mixture is then heated to 40° C. under agitation. Samples of 20 μL of the organic phase and 20 μL of the aqueous phase are then taken following the decantation of each phase after 0, 15, and 30 minutes and 1, 2, 4, 6 and 24 hours of reaction time. These aliquots are then diluted with 1.5 ml of methanol. Then 20 μL of decanol are added to the aqueous phase samples. All the extracts are then analysed by gas chromatography.

The composition of the organic phase is given in percentages determined from the chromatogram and is presented in Table 1 and FIG. 1.

TABLE 1
Composition of the organic phase:

| | Heptaldehyde | | | |
|---|---|---|---|---|
| Time (min) | (%) | HGA (%) | Glycerol (%) | Other (%) |
| 0 | 97% | 0% | 3% | 0% |
| 15 | 38% | 58% | 2% | 3% |
| 30 | 21% | 75% | 1% | 3% |
| 60 | 12% | 84% | 1% | 3% |
| 120 | 9% | 87% | 1% | 3% |
| 240 | 7% | 88% | 1% | 3% |
| 360 | 6% | 90% | 1% | 2% |
| 1440 | 3% | 93% | 1% | 2% |

The content in glycerol of the aqueous phase has been determined on the basis of the glycerol/decanol area ratio obtained at time 0 and taken as reference (100%). Thus, this same ratio calculated at 15, and 30 minutes, 1, 2, 4, 6, and 24 hours is expressed as a percentage in respect of the initial ratio.

The theoretical calculation of the variation in glycerol concentration in the aqueous phase resulted in an initial/final concentration ratio of 82%. For this calculation, a glycerol molar excess of 3 was considered and therefore a maximum conversion of 33%. The results of the experiment are shown in FIG. 1.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this present disclosure are described herein, including the best mode known to the inventors for carrying out the present disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, this present disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the subject matter disclosed herein are illustrative of the principles of the present disclosure. Other modifications that may be employed are within the scope of the subject matter. Thus, by way of example, but not of limitation, alternative configurations of the present disclosure may be utilized in accordance with the teachings herein. Accordingly, the present disclosure is not limited to that precisely as shown and described.

1. A method of synthesizing cyclic acetals comprising:
reacting at least one carbonyl-functionalized compound selected from the group consisting of aldehydes, ketones, and linear acetals, and combinations thereof,
with a polyol in a concentrated aqueous solution containing an acid catalyst to produce at least one cyclic acetal; and
simultaneously separating said at least one cyclic acetal;
wherein said carbonyl-functionalized compound yields at least one cyclic acetal having a water solubility of less than 20000 mg/kg at room temperature.

2. The method of claim 1, wherein the polyol concentration in said concentrated aqueous solution is greater than or equal to 20% by weight.

3. The method of claim 1, wherein said catalyst is a homogeneous catalyst comprising an acid selected from the group consisting of hydrochloric acid, nitric acid, sulphuric acid, methane sulphonic acid, paratoluenesulphonic acid, triflic acid, and oxalic acid; and combinations thereof.

4. The method of claim 1, wherein said catalyst is a heterogeneous catalyst comprising a solid acid having a Hammett Hₒ acidity which is less than +2.

5. The method of claim 1, wherein said carbonyl-functionalized compound is an aldehyde or a ketone with a number of atoms between 4 and 12.

6. The method of claim 1, wherein said carbonyl-functionalized compound has a water solubility which is less than 20000 mg/kg.

7. The method of claim 1, wherein said carbonyl-functionalized compound is n-heptanal.

8. The method of claim 1, wherein said reacting and separating are performed in a system of reactors in stages which increase the relative concentration of aldehyde or ketone from one stage to the next stage.

9. The method of claim 1, wherein said catalyst is a heterogeneous catalyst comprising a solid acid having a Hammet Hₒ acidity which is less than 0.

10. The method of claim 1, wherein said carbonyl-functionalized compound is an aldehyde or ketone with a number of atoms between 5 and 9.

11. The method of claim 1, wherein said carbonyl functionalized compound is: 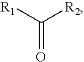
wherein R₁ and R₂ can be same or different, and each is H, a linear or branched alkyl radical, saturated or unsaturated hydrocarbon, or C₁-C₂₂ cyclic or aromatic radical.

12. The method of claim 1, wherein said polyol is: 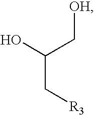
wherein R₃ is H, a linear or branched alkyl radical, saturated or unsaturated hydrocarbon, or C₁-C₂₂ cyclic or aromatic radical.

13. The method of claim 1, wherein said polyol is: 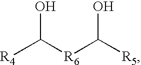
wherein R4 and R5 can be same or different, and each is H, a linear or branched alkyl radical, saturated or unsaturated hydrocarbon, or C₁-C₂₂ cyclic or aromatic radical; and
R₆ is a linear or branched alkyl radical, saturated or unsaturated hydrocarbon, or C₁-C₂₂ cyclic or aromatic radical.

14. The method of claim 1, wherein said polyol is:

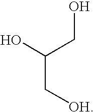

15. The method of claim 3, wherein said reactor is an agitated reactor.
